(19) United States
(12) Patent Application Publication
(10) Pub. No.: US 2023/0045078 A1
(43) Pub. Date: Feb. 9, 2023

(54) SYSTEMS AND METHODS FOR AUDIO PROCESSING AND ANALYSIS OF MULTI-DIMENSIONAL STATISTICAL SIGNATURE USING MACHINE LEARING ALGORITHMS

(71) Applicant: Aural Analytics, Inc., Scottsdale, AZ (US)

(72) Inventors: Visar BERISHA, Tempe, AZ (US); Julie LISS, Scottsdale, AZ (US); Daniel JONES, Phoenix, AZ (US); Shira HAHN, Tempe, AZ (US)

(21) Appl. No.: 17/759,088

(22) PCT Filed: Jan. 22, 2021
(86) PCT No.: PCT/US2021/014754

§ 371 Date: Jul. 19, 2022

Related U.S. Application Data
(60) Provisional application No. 62/964,642, filed on Jan. 22, 2020.
(60) Provisional application No. 62/964,646, filed on Jan. 22, 2020.

(51) Int. Cl. A61B 5/00; A61B 5/08; A61B 7/00; G10L 25/66; G10L 15/22; G10L 15/02

(52) U.S. Cl. CPC A61B 5/4803; A61B 5/0803; A61B 5/6898; A61B 5/742; A61B 5/486; A61B 5/7267; A61B 7/003; G10L 25/66; G10L 15/22; G10L 15/02; A61B 2562/0204

(57) ABSTRACT

Disclosed herein are systems, devices, and methods for evaluating or analyzing complex audio signals using multi-dimensional statistical signatures and machine learning algorithms. One advantage of the present disclosure is the ability for remote evaluation of respiratory tract health using speech analysis. The need for remote collection capabilities that can sensitively and reliably characterize respiratory tract function is particularly pertinent in view of the recent Covid-19 pandemic, which may adversely affect the health of individuals who could already be experiencing health problems with respiratory tract function.

### CROSS-REFERENCE TO RELATED APPLICATIONS

This application claims the benefit of U.S. Provisional Application Ser. No. 62/964,646, filed Jan. 22, 2020, and U.S. Provisional Application Ser. No. 62/964,642, filed Jan. 22, 2020, the contents of which are incorporated by reference herein in their entirety.

### BACKGROUND OF THE INVENTION

Respiratory tract health is associated with various health conditions. Respiratory function and vocal fold abnormalities or nasal congestion can be assessed using the gold standard measures of spirometry for respiratory function and clinical evaluation for vocal fold abnormalities or nasal congestion. However, these approaches require in-person evaluation which has the potential to expose the individual to pathogens in the hospital or clinic environment.

### SUMMARY OF THE INVENTION

Disclosed herein are systems and methods for evaluating or analyzing respiratory tract health or function using speech analysis. In some embodiments, respiratory tract health or function comprises congestion or congestion symptoms. In some embodiments, respiratory tract health or function comprises or is associated with smoking or smoking cessation status.

One advantage of the present disclosure is the ability for remote evaluation of respiratory tract health using speech analysis. The need for remote collection capabilities that can sensitively and reliably characterize respiratory tract function is particularly pertinent in view of the recent Covid-19 pandemic, which may adversely affect the health of individuals who could already be experiencing health problems with respiratory tract function.

Another advantage of the present disclosure is to provide an objective mechanism to evaluate and/or monitor respiratory tract health, congestion, smoking cessation, or other speech and/or respiration related conditions. The use of computer-implemented systems, devices, and methods to perform this analysis of speech or audio signals enables more efficient and objective results to be provided to the user or other relevant third parties (e.g., a healthcare provider).

The production of speech requires a controlled movement of air from the lungs through the respiratory tract. Following an inhalation, active (muscular) and passive (elastic recoil) forces push air from the lungs, through the bronchi and then the larynx. At the larynx, the ascending column of air sets the medialized vocal folds in vibration, which effectively chops the column into rapid puffs of air that create the sound of the voice. This excited airstream is filtered through the rest of the vocal tract, and is modulated by movements of articulators that change the shape and resonance characteristics of human oral and nasal cavities to produce speech. In this way, respiration is the power source for speech production.

Any reductions in the lungs' vital capacity or control, or structural impediments to the passage of air through the respiratory tract, are manifested in the speech signal. With regard to the lower airway, reductions in vital capacity (e.g., the amount of air that can be voluntarily exchanged) can be caused by muscular weakness, such as in ALS or spinal injury; or by conditions that interfere with the expansion of the lungs or bronchi, such as asthma, COPD and pneumonia. This can manifest as various conditions or symptoms such as low vocal loudness, only a few words uttered per breath, and increased pausing to inhale. Physical or functional barriers to airflow also can occur at the level of the larynx. Edema or paralysis of the vocal folds can reduce the size of the glottis, causing resistance to airflow ascending from the lungs and bronchi. This can manifest as poor vocal quality, and reduced modulation of pitch and loudness.

Dysfunction of vocal fold modulation, as with spasmodic dysphonia, can interfere with the appropriate passage of air and vocal fold vibration. This is evident by intermittent voice stoppages and poor vocal quality. With regard to the upper airway, nasal congestion impedes both the passage of air through the nasal cavity, and dampens of the nasal cavity's resonation properties. This interferes with the production of nasal consonants (e.g., “m” and “n”) that require air to flow through the nasal cavity and produce a nasal resonance. This causes nasal sounds to be produced more like their oral cognates (e.g., “m” and “d” sound more like “b” and “d”, respectively), and have the sound quality of hyponasality (not enough nasal resonance). These characteristics can be quantified acoustically as the precision of articulation of consonants and as the ratio of oral-to-nasal resonance in the speech.

Various changes can occur in the human body following smoking cessation. Exhaled nitric oxide levels can increase to nearly normal values within one week of smoking cessation. Macroscopic signs of chronic bronchitis (oedema, erythema and mucus) decrease within three months after smoking cessation, and totally disappear after about six months. The number of blood leukocytes falls almost immediately after smoking cessation. Macrophages in sputum and bronchoalveolar lavage fluid (BALF) are evident one to two months after smoking cessation, and reach normal levels at six months.

Detecting and tracking physiological changes secondary to smoking cessation is cumbersome. However, one advantage of the systems, and methods disclosed herein is the ability to detect changes to the human body attributable to smoking cessation that may manifest as changes to the phonatory and respiratory subsystems.

Regarding the respiratory subsystem, speaking occurs during exhalation in the context of most human language. This is because the outward flow of air from the lungs powers a human's speech apparatus. This column of air sets a person's vocal folds in vibration, which affects (e.g., chops) the airstream to produce the sound of the voice. This excited airstream is filtered through the rest of the vocal tract, and is modulated by movements of articulators that change the shape of human oral and nasal cavities.

Regarding the phonatory subsystem, vocal fold vibration (also known as phonation) provides the sound of a human voice. All vowels and many consonant sounds require voicing. For example, the difference between an “s” sound and a “z” sound is that the “z” is voiced. Healthy vocal folds can be set into vibration by air pressure generated by the lungs. Muscles in and around the vocal folds are modulated by commands from the brain to control voice pitch, loudness, and quality by changing the length, tension, and thickness of the vocal folds.

Changes to a human body after smoking cessation can be observed externally as changes in the sound of a person's speech. This is because the human speech production mechanism requires coordination between the respiratory and phonatory subsystems to produce healthy speech. These changes may be observed perceptually (e.g., by speech-language pathologists) and anecdotally are known to change.

Clinical assessments are predominantly conducted through subjective tests performed by speech-language pathologists (e.g. making subjective estimations of the amount of speech that can be understood, number of words correctly understood in a standard test battery, etc.). Perceptual judgments are easy to render and have strong face validity for characterizing speech deficits. Subjective tests, however, can be inconsistent and costly, often are not repeatable, and subjective judgments may be highly vulnerable to bias. In particular, repeated exposure to the same test subject (e.g., patient) over time can influence the assessment ratings generated by a speech-language pathologist. As such, there is an inherent ambiguity about whether the patient's intelligibility is confounded with increased familiarity with the patient's speech, as both may affect subjective assessment by the speech-language pathologist.

Disclosed herein are systems, devices, and methods that address a need to objectively evaluate speech parameters that tap into respiratory function and vocal quality, with emphasis on changes that occur after smoking cessation, such as to allow individuals who have stopped smoking to track physiological changes longitudinally.

Disclosed herein is a device for assessing speech changes resulting from respiratory tract function, the device comprising: audio input circuitry configured to provide an input signal that is indicative of speech provided by a user; signal processing circuitry configured to: receive the input signal; process the input signal to generate an instantaneous multi-dimensional statistical signature of speech production abilities of the user, and compare the multi-dimensional statistical signature against one or more baseline statistical signatures of speech production ability derived or obtained from the user; and provide a speech change identification signal attributable to respiratory tract function of the user, based on the multi-dimensional statistical signature comparison; and a notification element coupled to the signal processing circuitry, the notification element configured to receive the speech change identification and provide at least one notification signal to the user. In some embodiments, the multi-dimensional statistical signature spans one or more of the following perceptual dimensions: articulation, prosodic variability, phonation changes, rate, and rate variation. In some embodiments, the signal processing circuitry is configured to process the input signal by measuring speech features represented in the input signal, the speech features comprising one or more of articulation rate, articulation entropy, vowel space area, energy decay slope, phonatory duration, and average pitch. In some embodiments, the signal processing circuitry is configured to compare the multi-dimensional statistical signature against the one or more baseline statistical signatures of speech production ability by comparing each speech feature to a corresponding baseline speech feature of the one or more baseline statistical signatures of speech production ability. In some embodiments, the signal processing circuitry is configured to process the input signal utilizing the input signal and additional data comprising one or more of sensor data, a time of day, an ambient light level, a device usage pattern of the user, or a user input. In some embodiments, the signal processing circuitry is configured to process the input signal by selecting or adjusting the one or more baseline statistical signatures of speech production ability based on the additional data. In some embodiments, the device is a mobile computing device operating an application for assessing speech changes resulting from respiratory tract function. In some embodiments, the application queries the user periodically to provide a speech sample from which the input signal is derived. In some embodiments, the application facilitates the user spontaneously providing a speech sample from which the input signal is derived. In some embodiments, the application passively detects changes in speech patterns of the user and initiates generation of the instantaneous multi-dimensional statistical signature of speech production abilities of the user. In some embodiments, the notification element comprises a display. In some embodiments, the signal processing circuitry is further configured to cause the display to prompt the user to provide a speech sample from which the input signal is derived. In some embodiments, the at least one notification signal comprises a display notification instructing the user to take action to relieve symptoms associated with respiratory tract function.

Disclosed herein is a method for assessing speech changes resulting from respiratory tract function, the method comprising: receiving an input signal that is indicative of speech provided by a user; extracting a multi-dimensional statistical signature of speech production abilities of the user from the input signal; comparing the multi-dimensional statistical signature against one or more baseline statistical signatures of speech production ability; and providing a speech change identification signal attributable to respiratory tract function of the user, based on the multi-dimensional statistical signature comparison. In some embodiments, the one or more baseline statistical signatures of speech production ability are derived or obtained from the user. In some embodiments, the one or more baseline statistical signatures of speech production ability are at least partially based on normative acoustic data from a database. In some embodiments, the comparing the multi-dimensional statistical signature against the one or more baseline statistical signatures of speech production ability comprises applying a machine learning algorithm to the multi-dimensional statistical signature. In some embodiments, the machine learning algorithm is trained with past comparisons for other users. In some embodiments, extracting the multi-dimensional statistical signature of speech production abilities of the user from the input signal comprises measuring speech features across one or more of the following perceptual dimensions: articulation, prosodic variability, phonation changes, rate, and rate variation; and comparing the multi-dimensional statistical signature against the one or more baseline statistical signatures of speech production ability comprises comparing each speech feature to a corresponding baseline speech feature of the one or more baseline statistical signatures of speech production ability.

Disclosed herein is a non-transitory computer readable storage medium which, when executed by a computer, causes the computer to: receive an input signal that is indicative of speech provided by a user; extract a multi-dimensional statistical signature of speech production abilities of the user from the input signal; compare the multi-dimensional statistical signature against one or more baseline statistical signatures of speech production ability; and provide a speech change identification signal attributable to respiratory tract function of the user, based on the multi-dimensional statistical signature comparison.

Disclosed herein is a device for assessing speech production and respiration changes after smoking cessation, the device comprising: audio input circuitry configured to provide an input signal that is indicative of speech and respiration provided by a user; signal processing circuitry configured to: receive the input signal; process the input signal to generate an instantaneous multi-dimensional statistical signature of speech production and respiration abilities of the user, and compare the multi-dimensional statistical signature against one or more baseline statistical signatures of speech production and respiration ability derived or obtained from the user; and provide a speech production and respiration change identification signal based on the multi-dimensional statistical signature comparison; and a notification element coupled to the signal processing circuitry, the notification element configured to receive the speech production and respiration identification signal and provide at least one notification signal to the user. In some embodiments, the multi-dimensional statistical signature spans one or more of the following perceptual dimensions: articulation, prosodic variability, phonation changes, rate, and rate variation. In some embodiments, the signal processing circuitry is configured to process the input signal by measuring speech features represented in the input signal, the speech features comprising one or more of speaking rate, articulation rate, articulation entropy, vowel space area, energy decay slope, phonatory duration, and average pitch. In some embodiments, the signal processing circuitry is configured to compare the multi-dimensional statistical signature against the one or more baseline statistical signatures of speech production ability by comparing each speech feature to a corresponding baseline speech feature of the one or more baseline statistical signatures of speech production ability. In some embodiments, the signal processing circuitry is configured to process the input signal utilizing the input signal and additional data comprising one or more of sensor data, a time of day, an ambient light level, a device usage pattern of the user, or a user input. In some embodiments, the signal processing circuitry is configured to process the input signal by selecting or adjusting the one or more baseline statistical signatures of speech production ability based on the additional data. In some embodiments, the device is a mobile computing device operating an application for assessing speech production and respiration changes after smoking cessation. In some embodiments, the application queries the user periodically to provide a speech sample from which the input signal is derived. In some embodiments, the application facilitates the user spontaneously providing a speech sample from which the input signal is derived. In some embodiments, the application passively detects changes in speech patterns of the user and initiates generation of the instantaneous multi-dimensional statistical signature of speech production abilities of the user. In some embodiments, the notification element comprises a display. In some embodiments, the signal processing circuitry is further configured to cause the display to prompt the user to provide a speech sample from which the input signal is derived.

Disclosed herein is a method for assessing speech production and respiration changes after smoking cessation, the method comprising: receiving an input signal that is indicative of speech production and respiration provided by a user; extracting a multi-dimensional statistical signature of speech production and respiration abilities of the user from the input signal; comparing the multi-dimensional statistical signature against one or more baseline statistical signatures of speech production and respiration ability; and providing a speech production and respiration change identification signal attributable to smoking cessation based on the multi-dimensional statistical signature comparison. In some embodiments, the one or more baseline statistical signatures of speech production and respiration abilities are derived or obtained from the user. In some embodiments, the one or more baseline statistical signatures of speech production and respiration abilities are at least partially based on normative acoustic data from a database. In some embodiments, the comparing the multi-dimensional statistical signature against the one or more baseline statistical signatures of speech production and respiration abilities comprises applying a machine learning algorithm to the multi-dimensional statistical signature. In some embodiments, the machine learning algorithm is trained with past comparisons for other users. In some embodiments, extracting the multi-dimensional statistical signature of speech production abilities of the user from the input signal comprises measuring speech features across one or more of the following perceptual dimensions: articulation precision, respiratory support, nasality, prosody, and phonatory control; and comparing the multi-dimensional statistical signature against the one or more baseline statistical signatures of speech production ability comprises comparing each speech feature to a corresponding baseline speech feature of the one or more baseline statistical signatures of speech production ability.

Disclosed herein is a non-transitory computer readable storage medium which, when executed by a computer, causes the computer to: receive an input signal that is indicative of speech production and respiration provided by a user; extract a multi-dimensional statistical signature of speech production and respiration abilities of the user from the input signal; compare the multi-dimensional statistical signature against one or more baseline statistical signatures of speech production and respiration abilities; and provide a speech production and respiration change identification signal attributable to smoking cessation of the user, based on the multi-dimensional statistical signature comparison. In some embodiments, the computer is a smartphone.

Disclosed herein is a device for assessing speech changes resulting from congestion state, the device comprising: audio input circuitry configured to provide an input signal that is indicative of speech provided by a user; signal processing circuitry configured to: receive the input signal; process the input signal to generate an instantaneous multi-dimensional statistical signature of speech production abilities of the user, and compare the multi-dimensional statistical signature against one or more baseline statistical signatures of speech production ability derived or obtained from the user; and provide a speech change identification signal attributable to congestion state of the user, based on the multi-dimensional statistical signature comparison; and a notification element coupled to the signal processing circuitry, the notification element configured to receive the speech change identification and provide at least one notification signal to the user. In some embodiments, the multi-dimensional statistical signature spans one or more of the following perceptual dimensions: articulation, prosodic variability, phonation changes, rate, and rate variation. In some embodiments, the signal processing circuitry is configured to process the input signal by measuring speech features represented in the input signal, the speech features comprising one or more of articulation rate, articulation entropy, vowel space area, energy decay slope, phonatory duration, and average pitch. In some embodiments, the signal processing circuitry is configured to compare the multi-dimensional statistical signature against the one or more baseline statistical signatures of speech production ability by comparing each speech feature to a corresponding baseline speech feature of the one or more baseline statistical signatures of speech production ability. In some embodiments, the signal processing circuitry is configured to process the input signal utilizing the input signal and additional data comprising one or more of sensor data, a time of day, an ambient light level, a device usage pattern of the user, or a user input. In some embodiments, the signal processing circuitry is configured to process the input signal by selecting or adjusting the one or more baseline statistical signatures of speech production ability based on the additional data. In some embodiments, the device is a mobile computing device operating an application for assessing speech changes resulting from congestion state. In some embodiments, the application queries the user periodically to provide a speech sample from which the input signal is derived. In some embodiments, the application facilitates the user spontaneously providing a speech sample from which the input signal is derived. In some embodiments, the application passively detects changes in speech patterns of the user and initiates generation of the instantaneous multi-dimensional statistical signature of speech production abilities of the user. In some embodiments, the notification element comprises a display. In some embodiments, the signal processing circuitry is further configured to cause the display to prompt the user to provide a speech sample from which the input signal is derived. In some embodiments, the at least one notification signal comprises a display notification instructing the user to take action to relieve congestion symptoms.

Disclosed herein is a method for assessing speech changes resulting from congestion state, the method comprising: receiving an input signal that is indicative of speech provided by a user; extracting a multi-dimensional statistical signature of speech production abilities of the user from the input signal; comparing the multi-dimensional statistical signature against one or more baseline statistical signatures of speech production ability; and providing a speech change identification signal attributable to congestion state of the user, based on the multi-dimensional statistical signature comparison. In some embodiments, the one or more baseline statistical signatures of speech production ability are derived or obtained from the user. In some embodiments, the one or more baseline statistical signatures of speech production ability are at least partially based on normative acoustic data from a database. In some embodiments, the comparing the multi-dimensional statistical signature against the one or more baseline statistical signatures of speech production ability comprises applying a machine learning algorithm to the multi-dimensional statistical signature. In some embodiments, the machine learning algorithm is trained with past comparisons for other users. In some embodiments, extracting the multi-dimensional statistical signature of speech production abilities of the user from the input signal comprises measuring speech features across one or more of the following perceptual dimensions: articulation, prosodic variability, phonation changes, rate, and rate variation; and comparing the multi-dimensional statistical signature against the one or more baseline statistical signatures of speech production ability comprises comparing each speech feature to a corresponding baseline speech feature of the one or more baseline statistical signatures of speech production ability.

Disclosed herein is a non-transitory computer readable storage medium which, when executed by a computer, causes the computer to: receive an input signal that is indicative of speech provided by a user; extract a multi-dimensional statistical signature of speech production abilities of the user from the input signal; compare the multi-dimensional statistical signature against one or more baseline statistical signatures of speech production ability; and provide a speech change identification signal attributable to congestion state of the user, based on the multi-dimensional statistical signature comparison.

Disclosed herein is a system for performing multi-dimensional analysis of complex audio signals using machine learning, the system comprising: audio input circuitry configured to provide an input signal that is indicative of speech provided by a user; signal processing circuitry configured to: receive the input signal; perform audio pre-processing on the input signal, wherein the pre-processing comprises: background noise estimation; diarization analysis using a Gaussian mixture model to identify a plurality of distinct speakers from the input signal; and transcription of the input signal using a speech recognition algorithm; generate an alignment of transcribed text with the plurality of distinct speakers based on the audio pre-processing; process the input signal to generate an instantaneous multi-dimensional statistical signature of speech production abilities, the multi-dimensional statistical signature comprising a plurality of features extracted from the input signal; evaluate the multi-dimensional statistical signature against one or more baseline statistical signatures of speech production ability using a deep learning convolutional neural network trained using a training dataset, thereby generating a speech change identification signal; and a notification element coupled to the signal processing circuitry, the notification element configured to receive the speech change identification and transmit or provide at least one notification signal to the user.

Disclosed herein are systems, devices, methods, and non-transitory computer readable storage medium for carrying out any of the speech or audio processing and/or analyses of the present disclosure. Any embodiments specifically directed to a system, a device, a method, or a non-transitory computer readable storage medium is also contemplated as being implemented in any alternative configuration such as a system, a device, a method, or a non-transient/non-transitory computer readable storage medium.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic diagram depicting a system for assessing parameters of speech resulting from a health or physiological state or change. The system may be configured for evaluating congestion/decongestion and/or smoking/smoking cessation.

FIG. 2 is a flow diagram illustrating a process for assessing speech changes resulting from respiration tract function (e.g., congestion).

FIG. 3 is a flow diagram illustrating a process for assessing speech changes resulting from smoking or smoking cessation.

FIG. 4 is a flow diagram illustrating a series of audio pre-processing steps, feature extraction, and analysis according to some embodiments of the present disclosure.

### DETAILED DESCRIPTION OF THE INVENTION

Disclosed herein are systems, devices, and methods for objectively evaluating speech parameter changes secondary to one or more respiratory tract conditions. In some embodiments, the one or more respiratory tract conditions comprises congestion or decongestion.

Disclosed herein are systems, devices, and methods for objectively evaluating speech parameter changes secondary to smoking or smoking cessation. In some embodiments, one or more respiratory tract conditions comprises respiration changes that occur after smoking cessation.

The information set forth herein enable those skilled in the art to practice the embodiments and illustrate the best mode of practicing the embodiments. Upon reading the description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The systems and methods disclosed herein enables objective identification and/or prediction of physiological changes, states, or conditions associated with speech production and/or respiration change. This improved approach provides more effective and convenient detection of such physiological states (e.g., congestion episodes, relapse in smoking cessation, etc.) and rapid therapeutic intervention. In certain embodiments, an application running in the background of a mobile phone may passively detect subtle changes in the speech patterns of an individual suffering from congestion or who has resumed smoking during phone calls, by periodically generating a multi-dimensional statistical signature of a user's speech production abilities and comparing the signature against one or more baseline signatures. When changes in a user's speech production abilities consistent with a change in congestion state are detected, the phone may notify the user and instruct the user to take appropriate action (e.g., adjusting activity and/or taking medication).

In certain embodiments, the periodic generation of multi-dimensional statistical signatures of a user's speech production abilities for a user undergoing a pharmaceutical treatment regimen for treating nasal and/or sinus congestion may be used to assess efficacy of the treatment regimen.

Disclosed herein is a panel of measures (e.g., features subject to measurement via speech or audio analysis) for assessing respiration, phonation, articulation, and prosody (and optionally velopharyngeal function) from continuous speech. In certain embodiments, such measures are implemented in a mobile application with a user interface, algorithms for processing the speech, visualization to track these changes, or any combination thereof. Detecting and tracking physiological changes secondary to smoking cessation is cumbersome. Speech analysis provides a novel and unobtrusive approach to this detection and tracking since the data can be collected frequently and using a participant's personal electronic device(s) (e.g., smartphone, tablet computer, etc.).

In some embodiments, a mobile device operating a mobile application is used to collect speech samples from an individual. The individual may be subject to experiencing changes in congestion state. In some cases, the individual has recently stopped smoking. These speech samples can be either actively or passively collected. Algorithms that analyze speech based on statistical signal processing are used to extract several parameters.

In certain embodiments, a speech sample is elicited from a user (e.g., periodically or on demand), and a multi-dimensional statistical signature of the user's current speech production abilities is generated for the speech sample (e.g., based on the speech features). The multi-dimensional statistical signature can be compared against one or more baseline statistical signatures of speech production ability. The baseline statistical signatures can be derived or obtained from the user in some examples, and can alternatively or additionally be based on normative data from a database (e.g., other users). The multi-dimensional statistical signature can refer to the combination of feature measurements that are used to evaluate a particular composite (e.g., specific measurements of pause rate, loudness, loudness decay that make up the signature for evaluating respiration).

In certain embodiments, complementary feature sets that represent physical characteristics of speech may be extracted from participant speech recordings, including one or more of the following items 1-5 described below.

1. Articulation precision, including vowel precision and consonant precision.

2. Respiratory support, including perceptual loudness decay and phonatory duration.

3. Nasality (e.g., measure of velopharyngeal function), including low-frequency energy distribution, as well as low-frequency/high-frequency ratio.

4. Prosody, including speaking rate, speaking rate variability, pause rate, pause rate variability, articulation rate, articulation rate variability, mean F0 and F0 variability.

5. Phonatory control, including pitch control, loudness control, and voice quality.

In certain embodiments, speech features subject to being measured and analyzed comprise one or more of speaking rate, articulation rate, articulation entropy, vowel space area, energy decay slope, phonatory duration, and average pitch.

In certain embodiments, a multi-dimensional statistical signature spans one or more of the following perceptual dimensions: articulation, prosodic variability, phonation changes, rate, and rate variation.

Additionally provided are visualization tools to permit the foregoing parameters to be tracked longitudinally and to provide insight into the physiological changes that occur secondarily to smoking cessation. A visualization tool allows an individual user or a medical care provider to track these changes in an interpretable way.

In certain embodiments, speech features for smokers, non-smokers, and individuals who have ceased smoking may be compared, with average values and standard deviations of speech feature sets being subject to comparison.

Embodiments disclosed herein provide an objective tool for evaluation of several speech parameters that tap into respiratory function and vocal quality, tailored for sensitively detecting changes in individuals immediately after smoking cessation.

In certain embodiments, p-values may be utilized to compare various speech features (e.g., speaking rate, pause rate, articulation rate, articulation entropy, vowel space area, energy decay slope, phonatory duration, and average pitch), including comparisons within a smoking group, non-smoking group, and smoking cessation group. In certain embodiments, data for participants may be gathered and organized by time window since smoking cessation. Other physiological parameters correlated to cessation of smoking may also be gathered and correlated to speech parameters. Speech patterns and respiratory abilities of different groups may be compared.

Systems and Methods for Assessing Speech Production and/or Respiration Changes

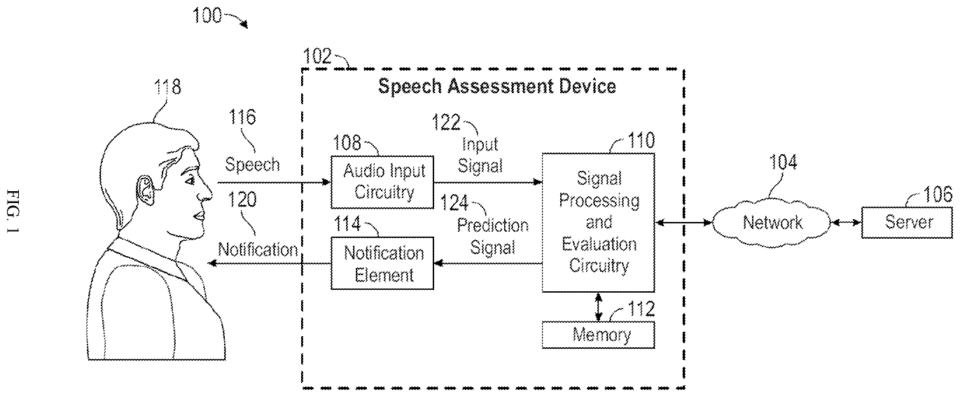

FIG. 1 is a diagram of a system 100 for assessing speech production and respiration changes, the system comprising a speech production and respiration assessment device 102, a network 104, and a server 106. The speech production and respiration assessment device 102 comprises audio input circuitry 108, signal processing circuitry 110, memory 112, and at least one notification element 114. In certain embodiments, the signal processing circuitry 110 may include, but not necessarily be limited to, audio processing circuitry. In some cases, the signal processing circuitry is configured to provide at least one speech assessment signal (e.g., generated outputs based on algorithmic/model analysis of input feature measurements) based on characteristics of speech provided by a user (e.g., speech or audio stream or data). The audio input circuitry 108, notification element(s) 114, and memory 112 may be coupled with the signal processing circuitry 110 via wired connections, wireless connections, or a combination thereof. The speech production and respiration assessment device 102 may further comprise a smartphone, a smartwatch, a wearable sensor, a computing device, a headset, a headband, or combinations thereof. The speech production and respiration assessment device 102 may be configured to receive speech 116 from a user 118 and provide a notification 120 to the user 118 based on processing the speech 116 and any respiration signals to assess changes in speech and respiration attributable to smoking cessation.

The audio input circuitry 108 may comprise at least one microphone. In certain embodiments, the audio input circuitry 108 may comprise a bone conduction microphone, a near field air conduction microphone array, or a combination thereof. The audio input circuitry 108 may be configured to provide an input signal 122 that is indicative of the speech 116 provided by the user 118 to the signal processing circuitry 110. The input signal 122 may be formatted as a digital signal, an analog signal, or a combination thereof. In certain embodiments, the audio input circuitry 108 may provide the input signal 122 to the signal processing circuitry 110 over a personal area network (PAN). The PAN may comprise Universal Serial Bus (USB), IEEE 1394 (FireWire) Infrared Data Association (IrDA), Bluetooth, ultra-wideband (UWB), Wi-Fi Direct, or a combination thereof. The audio input circuitry 108 may further comprise at least one analog-to-digital converter (ADC) to provide the input signal 122 in digital format.

The signal processing circuitry 110 may comprise a communication interface (not shown) coupled with the network 104 and a processor (e.g., an electrically operated microprocessor (not shown) configured to execute a pre-defined and/or a user-defined machine readable instruction set, such as may be embodied in computer software) configured to receive the input signal 122. The communication interface may comprise circuitry for coupling to the PAN, a local area network (LAN), a wide area network (WAN), or a combination thereof. The processor may be configured to receive instructions (e.g., software, which may be periodically updated) for extracting a multi-dimensional statistical signature of speech production abilities of the user 118 that spans multiple perceptual dimensions. Such perceptual dimensions may include any one or more of (A) articulation (providing measures of articulatory precision and articulator control); (B) prosodic variability (providing measures of intonational variation over time); (C) phonation changes (providing measures related to pitch and voicing); and (D) rate and rate variation (providing measures related to speaking rate and how it varies). In some cases, perceptual dimension refers to a composite.

Extracting the multi-dimensional statistical signature of speech production and respiratory abilities of the user 118 can include measuring one or more of the speech features described above. For example, the speech production and respiratory features may include one or more of articulation precision, respiratory support, nasality, prosody, and phonatory control, as described herein above.

Machine learning algorithms based on these acoustic measures may be used assess changes in speech and respiration attributable to smoking cessation. In certain embodiments, machine learning algorithms may use clusters of acoustic measures derived from a speech input signal and produce a speech and respiration change identification signal. In certain embodiments, an instantaneous multi-dimensional statistical signature may be normalized and/or compared against one or more baseline statistical signatures of speech production ability derived or obtained from the same subject (optionally augmented with statistical signatures and/or other information obtained from different subjects) to produce a speech and respiration change identification signal.

In certain embodiments, such machine learning algorithms (or other signal processing approaches) may compare the multi-dimensional statistical signature against one or more baseline statistical signatures of speech production and respiratory abilities by comparing each of several features (e.g., articulation precision, respiratory support, nasality, prosody, and phonatory control) to corresponding baseline speech and respiration feature of one or more baseline statistical signatures of speech production and respiration abilities. In certain embodiments, the machine learning algorithms may also take into account additional data, such as sensor data (e.g., from an accelerometer or environmental sensor), a time of day, an ambient light level, and/or a device usage pattern of the user.

In some cases, additional data can include input by the user, that may occur after smoking cessation. Such additional data may be part of the multi-dimensional statistical signature or may be used in analyzing the multi-dimensional statistical signature. For example, the additional data may be used to select or adjusting the baseline statistical signatures of speech and respiration abilities.

In certain embodiments, the processor may comprise an ADC to convert the input signal 122 to digital format. In other embodiments, the processor may be configured to receive the input signal 122 from the PAN via the communication interface. The processor may further comprise level detect circuitry, adaptive filter circuitry, voice recognition circuitry, or a combination thereof. The processor may be further configured to process the input signal 122 using a multi-dimensional statistical signature and/or clusters of acoustic measures derived from a speech input signal and produce a speech and respiration assessment signal, and provide a speech production and respiration change identification signal 124 to the notification element 114. The speech production and respiration change identification signal 124 may be in a digital format, an analog format, or a combination thereof. In certain embodiments, the speech production and respiration change identification signal 124 may comprise one or more of an audible signal, a visual signal, a vibratory signal, or another user-perceptible signal. In certain embodiments, the processor may additionally or alternatively provide the speech production and respiration change identification signal 124 over the network 104 via a communication interface.

The processor may be further configured to generate a record indicative of the speech production and respiration change identification signal 124. The record may comprise a sample identifier and/or an audio segment indicative of the speech 116 provided by the user 118. In certain embodiments, the user 118 may be prompted to provide current symptoms or other information about their current well-being to the speech production and respiration change assessment device 102 for assessing speech production and respiration changes. Such information may be included in the record, and may further be used to aid in identification or prediction of changes in congestion state.

The record may further comprise a location identifier, a time stamp, a physiological sensor signal (e.g., heart rate, blood pressure, temperature, or the like), or a combination thereof being correlated to and/or contemporaneous with the speech and respiration change identification signal 124. The location identifier may comprise a Global Positioning System (GPS) coordinate, a street address, a contact name, a point of interest, or a combination thereof. In certain embodiments, a contact name may be derived from the GPS coordinate and a contact list associated with the user 118. The point of interest may be derived from the GPS coordinate and a database including a plurality of points of interest. In certain embodiments, the location identifier may be a filtered location for maintaining the privacy of the user 118. For example, the filtered location may be “user's home”, “contact's home”, “vehicle in transit”, “restaurant”, or “user's work”. In certain embodiments, the record may include a location type, wherein the location identifier is formatted according to the location type.

The processor may be further configured to store the record in the memory 112. The memory 112 may be a non-volatile memory, a volatile memory, or a combination thereof. The memory 112 may be wired to the signal processing circuitry 110 using an address/data bus. In certain embodiments, the memory 112 may be portable memory coupled with the processor.

In certain embodiments, the processor may be further configured to send the record to the network 104, wherein the network 104 sends the record to the server 106. In certain embodiments, the processor may be further configured to append to the record a device identifier, a user identifier, or a combination thereof. The device identifier may be unique to the speech production and respiration change assessment device 102. The user identifier may be unique to the user 118. The device identifier and the user identifier may be useful to a medical treatment professional and/or researcher, wherein the user 118 may be a patient of the medical treatment professional.

The network 104 may comprise a PAN, a LAN, a WAN, or a combination thereof. The PAN may comprise USB, IEEE 1394 (FireWire) IrDA, Bluetooth, UWB, Wi-Fi Direct, or a combination thereof. The LAN may include Ethernet, 802.11 WLAN, or a combination thereof. The network 104 may also include the Internet.

The server 106 may comprise a personal computer (PC), a local server connected to the LAN, a remote server connected to the WAN, or a combination thereof. In certain embodiments, the server 106 may be a software-based virtualized server running on a plurality of servers.

In certain embodiments, at least some signal processing tasks may be performed via one or more remote devices (e.g., the server 106) over the network 104 instead of within a speech production and respiration change assessment device 102 that houses the audio input circuitry 108.

In certain embodiments, congestion state identification or prediction based on audio input signals may be augmented with signals indicative of physiological state and/or activity level of a user (e.g., heart rate, blood pressure, temperature, etc.). For example, audio input signals may be affected by activity level and/or physiological state of a user. In certain embodiments, a multi-dimensional statistical signature of speech production abilities obtained from a user may be normalized based on physiological state and/or activity level of the user before comparison is made against one or more baseline statistical signatures of speech production ability derived or obtained from the user, to avoid false positive or false negative congestion state identification signals. In certain embodiments, the one or more baseline statistical signatures of speech production ability are at least partially based on normative acoustic data from a database. For example, the baseline statistical signature(s) may be produced by a machine learning algorithm trained with past data for other users.

In certain embodiments, a speech production and respiration change assessment device 102 may be embodied in a mobile application configured to run on a mobile computing device (e.g., smartphone, smartwatch) or other computing device. With a mobile application, speech samples can be collected remotely from patients and analyzed without requiring patients to visit a clinic. A user 118 may be periodically queried (e.g., two, three, four, five, or more times per day) to provide a speech sample. For example, the notification element 114 may be used to prompt the user 118 to provide speech 116 from which the input signal 122 is derived, such as through a display message or an audio alert. The notification element 114 may further provide instructions to the user 118 for providing the speech 116 (e.g., displaying a passage for the user 118 to read). In certain embodiments, the notification element 114 may request current symptoms or other information about the current well-being of the user 118 to provide additional data for analyzing the speech 116.

In addition, whenever a user feels a congestion episode coming on, the user may open the application and provide a speech sample (e.g., spontaneously provide a speech sample). In certain embodiments, data collection may take no longer than 2-3 minutes as users are asked to read a carefully designed passage (e.g., paragraph) that evaluates the user's ability to produce all of the phonemes in the user's native language (e.g., English). Restated, a user may be provided with one or more speaking prompts, wherein such prompts may be tailored to the type of speech (data) that clinicians are interested in capturing. Examples of speaking tasks that may be prompted by a user include unscripted speech, reciting scripted sentences, and/or holding a single tone as long as possible (phonating). In certain embodiments, data collection may take additional time. In certain embodiments, the speech production and respiration change identification device may passively monitor the user's speech, and if a change in speech patterns is detected initiate an analysis to generate the instantaneous multi-dimensional statistical signature of speech production abilities of the user.

In certain embodiments, a notification element may include a display (e.g., LCD display) that displays text and prompts the user to read the text. Each time the user provides a new sample using the mobile application, a multi-dimensional statistical signature of the user's speech production abilities may be automatically extracted. One or more machine-learning algorithms based on these acoustic measures may be implemented to aid in identifying and/or predicting a physiological change or other physiological condition or state associated with speech and/or respiration. Examples include changes in speech production and/or respiration ability associated with congestion state (e.g., change in congestion such as increased/decreased congestion or an overall congestion status), and smoking or smoking cessation (e.g., change in smoking state such as having ceased smoking for a period of time).

In certain embodiments, a speech production and respiration change identification signal 124 provided to the notification element 114 can instruct the user 118 to take action, for example, to relieve congestion symptoms in the event of a change in congestion state. Such actions may include adjusting the environment (e.g., informed by sensor data received by the mobile application), taking medicine or other treatments, and so on. In some examples, the instructions may be customized to the user 118 based on previously successful interventions.

In certain embodiments, a user may download a mobile application to a personal computing device (e.g., smartphone), optionally sign in to the application, and follow the prompts on a display screen. Once recording has finished, the audio data may be automatically uploaded to a secure server (e.g., a cloud server or a traditional server) where the signal processing and machine learning algorithms operate on the recordings.

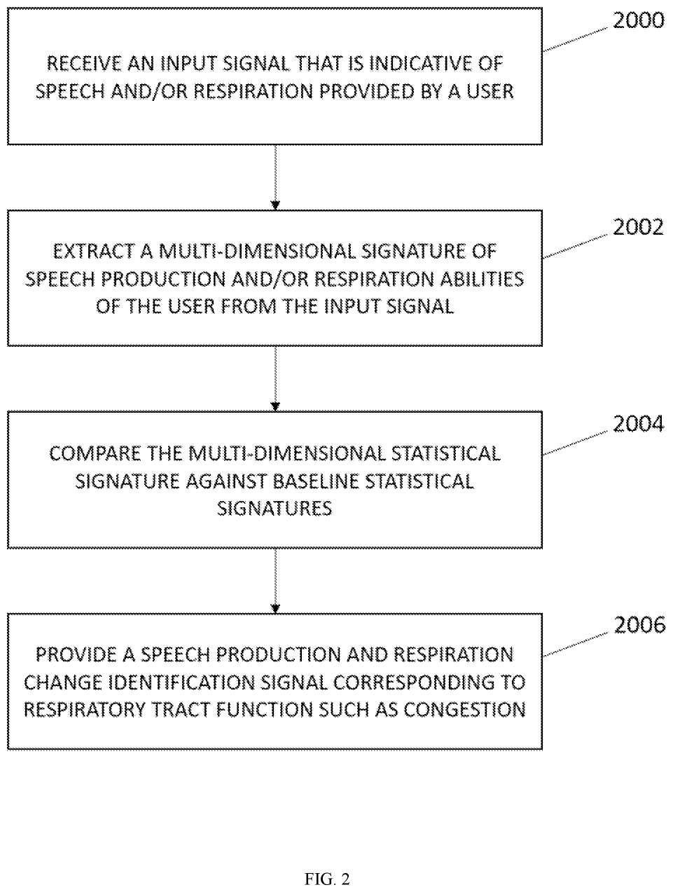

FIG. 2 is a flow diagram illustrating a process for assessing speech resulting from a physiological state or condition such as, for example, congestion state according to one embodiment. Other embodiments can include a state of smoking cessation. Specifically, FIG. 2 shows a flow diagram illustrating a process for assessing speech production and respiration changes resulting from or occurring after a physiological state or condition such as congestion state. The process may be performed by one or more components of the speech change assessment system 100 of FIG. 1, such as the device 102 or the server 106. The process begins at operation 2000, with receiving an input signal that is indicative of speech and respiration provided by a user. The process continues at operation 2002, with extracting a multi-dimensional statistical signature of speech production and respiration abilities of the user from the input signal. The process continues at operation 2004, with comparing the multi-dimensional statistical signature against one or more baseline statistical signatures of speech production and respiration ability. The process continues at operation 2006, with providing a speech production and respiration change identification signal attributable to the physiological change or state (e.g., congestion state of the user) based on the multi-dimensional statistical signature comparison.

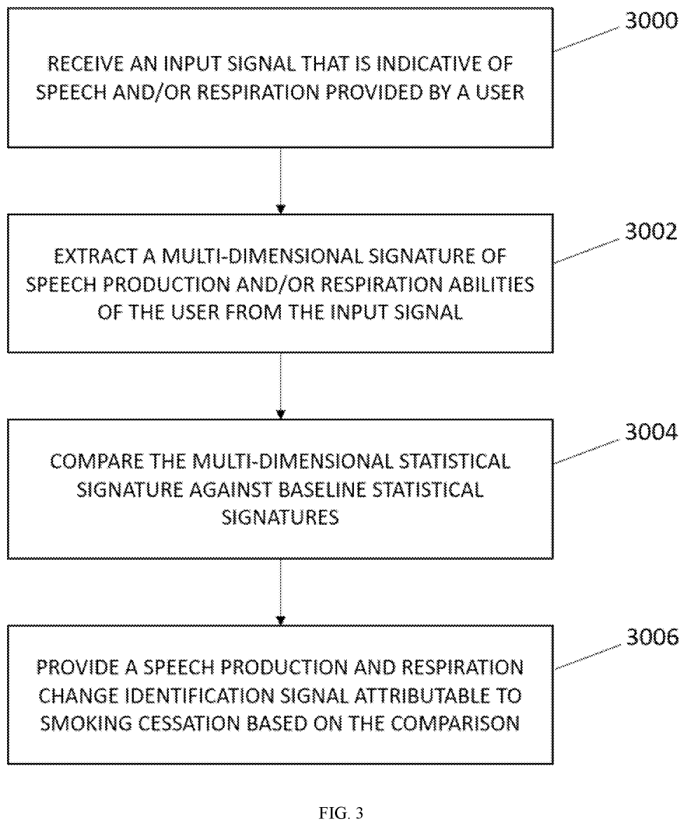

FIG. 3 is a flow diagram illustrating a process for assessing speech production and respiration changes resulting from or occurring after a physiological state or condition such as smoking/smoking cessation state. The process may be performed by one or more components of the speech change assessment system 100 of FIG. 1, such as the device 102 or the server 106. The process begins at operation 3000, with receiving an input signal that is indicative of speech and respiration provided by a user. The process continues at operation 3002, with extracting a multi-dimensional statistical signature of speech production and respiration abilities of the user from the input signal. The process continues at operation 3004, with comparing the multi-dimensional statistical signature against one or more baseline statistical signatures of speech production and respiration ability. The process continues at operation 3006, with providing a speech production and respiration change identification signal attributable to the physiological change or state (e.g., smoking/smoking cessation state of the user) based on the multi-dimensional statistical signature comparison.

Although the operations of FIG. 2 and FIG. 3 are illustrated in a series, this is for illustrative purposes and the operations are not necessarily order dependent. Some operations may be performed in a different order than that presented. Further, processes within the scope of this disclosure may include fewer or more steps than those illustrated in FIG. 2 and FIG. 3, respectively.

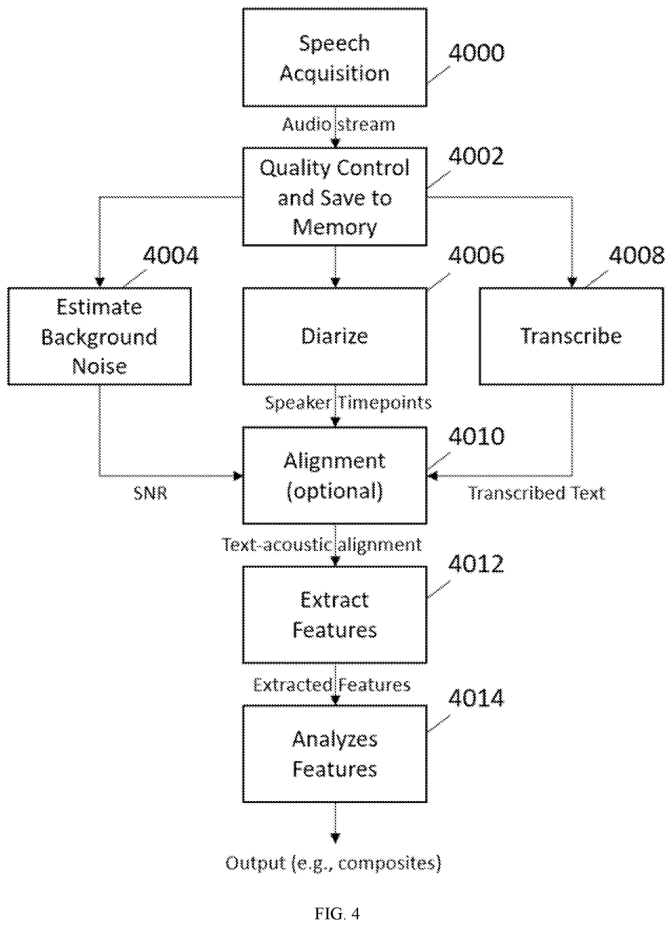

As shown in FIG. 4, the process for speech/language feature extraction and analysis can include one or more steps such as speech acquisition 4000, quality control 4002, background noise estimation 4004, diarization 4006, transcription 4008, optional alignment 4010, feature extraction 4012, and/or feature analysis 4014. In some embodiments, the systems, devices, and methods disclosed herein include a speech acquisition step. Speech acquisition 4000 can be performed using any number of audio collection devices. Examples include microphones or audio input devices on a laptop or desktop computer, a portable computing device such as a tablet, mobile devices (e.g., smartphones), digital voice recorders, audiovisual recording devices (e.g., video camera), and other suitable devices. In some embodiments, the speech or audio is acquired through passive collection techniques. For example, a device may be passively collecting background speech via a microphone without actively eliciting the speech from a user or individual. The device or software application implemented on the device may be configured to begin passive collection upon detection of background speech. Alternatively, or in combination, speech acquisition can include active elicitation of speech. For example, a mobile application implemented on the device may include instructions prompting speech by a user or individual. In some embodiments, the systems, devices, and methods disclosed herein utilize a dialog bot or chat bot that is configured to engage the user or individual in order to elicit speech. As an illustrative example, the bot may engage in a conversation with the user (e.g., via a graphic user interface such as a smartphone touchscreen or via an audio dialogue). Alternatively or in combination with a conversation, the bot may simply provide instructions to the user to perform a particular task (e.g., instructions to vocalize pre-written speech or sounds). In some cases, the speech or audio is not limited to spoken words, but can include nonverbal audio vocalizations made by the user or individual. For example, the user may be prompted with instructions to make a sound that is not a word for a certain duration.

In some embodiments, the systems, devices, and methods disclosed herein include a quality control step 4002. The quality control step may include an evaluation or quality control checkpoint of the speech or audio quality. Quality constraints may be applied to speech or audio samples to determine whether they pass the quality control checkpoint. Examples of quality constraints include (but are not limited to) signal to noise ratio (SNR), speech content (e.g., whether the content of the speech matches up to a task the user was instructed to perform), audio signal quality suitability for downstream processing tasks (e.g., speech recognition, diarization, etc.). Speech or audio data that fails this quality control assessment may be rejected, and the user asked to repeat or redo an instructed task (or alternatively, continue passive collection of audio/speech). Speech or audio data that passes the quality control assessment or checkpoint may be saved on the local device (e.g., user smartphone, tablet, or computer) and/or on the cloud. In some cases, the data is both saved locally and backed up on the cloud. In some embodiments, one or more of the audio processing and/or analysis steps are performed locally or remotely on the cloud.

In some embodiments, the systems, devices, and methods disclosed herein include background noise estimation 4004. Background noise estimation can include metrics such as a signal-to-noise ratio (SNR). SNR is a comparison of the amount of signal to the amount background noise, for example, ratio of the signal power to the noise power in decibels. Various algorithms can be used to determine SNR or background noise with non-limiting examples including data-aimed maximum-likelihood (ML) signal-to-noise ratio (SNR) estimation algorithm (DAML), decision-directed ML SNR estimation algorithm (DDML) and an iterative ML SNR estimation algorithm.

In some embodiments, the systems, devices, and methods disclosed herein perform audio analysis of speech/audio data stream such as speech diarization 4006 and speech transcription 4008. The diarization process can include speech segmentation, classification, and clustering. The speech or audio analysis can be performed using speech recognition and/or speaker diarization algorithms. Speaker diarization is the process of segmenting or partitioning the audio stream based on the speaker's identity. As an example, this process can be especially important when multiple speakers are engaged in a conversation that is passively picked up by a suitable audio detection/recording device. In some embodiments, the diarization algorithm detects changes in the audio (e.g., acoustic spectrum) to determine changes in the speaker, and/or identifies the specific speakers during the conversation. An algorithm may be configured to detect the change in speaker, which can rely on various features corresponding to acoustic differences between individuals. The speaker change detection algorithm may partition the speech/audio stream into segments. These partitioned segments may then be analyzed using a model configured to map segments to the appropriate speaker. The model can be a machine learning model such as a deep learning neural network. Once the segments have been mapped (e.g., mapping to an embedding vector), clustering can be performed on the segments so that they are grouped together with the appropriate speaker(s).

Techniques for diarization include using a Gaussian mixture model, which can enable modeling of individual speakers that allows frames of the audio to be assigned (e.g., using Hidden Markov Model). The audio can be clustered using various approaches. In some embodiments, the algorithm partitions or segments the full audio content into successive clusters and progressively attempts to combine the redundant clusters until eventually the combined cluster corresponds to a particular speaker. In some embodiments, algorithm begins with a single cluster of all the audio data and repeatedly attempts to split the cluster until the number of clusters that has been generated is equivalent to the number of individual speakers. Machine learning approaches are applicable to diarization such as neural network modeling. In some embodiments, a recurrent neural network transducer (RNN-T) is used to provide enhanced performance when integrating both acoustic and linguistic cues. Examples of diarization algorithms are publicly available (e.g., Google).

Speech recognition (e.g., transcription of the audio/speech) may be performed sequentially or together with the diarization. The speech transcript and diarization can be combined to generate an alignment of the speech to the acoustics (and/or speaker identity). In some cases, passive and active speech are evaluated using different algorithms. Standard algorithms that are publicly available and/or open source may be used for passive speech diarization and speech recognition (e.g., Google and Amazon open source algorithms may be used). Non-algorithmic approaches can include manual diarization. In some embodiments, diarization and transcription are not required for certain tasks. For example, the user or individual may be instructed or required to perform certain tasks such as sentence reading tasks or sustained phonation tasks in which the user is supposed to read a pre-drafted sentence(s) or to maintain a sound for an extended period of time. In such tasks, transcription may not be required because the user is being instructed on what to say. Alternatively, certain actively acquired audio may be analyzed using standard (e.g., non-customized) algorithms or, in some cases, customized algorithms to perform diarization and/or transcription. In some embodiments, the dialogue or chat bot is configured with algorithm(s) to automatically perform diarization and/or speech transcription while interacting with the user

In some embodiments, the speech or audio analysis comprises alignment 4010 of the diarization and transcription outputs. The performance of this alignment step may depend on the downstream features that need to be extracted. For example, certain features require the alignment to allow for successful extraction (e.g., features based on speaker identity and what the speaker said), while others do not. In some embodiments, the alignment step comprises using the diarization output to extract the speech from the speaker of interest. Standard algorithms may be used with non-limiting examples including Kaldi, gentle, Montreal forced aligner), or customized alignment algorithms (e.g., using algorithms trained with proprietary data).

In some embodiments, the systems, devices, and methods disclosed herein perform feature extraction 4012 from one or more of the SNR, diarization, and transcription outputs. One or more extracted features can be analyzed 4014 to predict or determine an output comprising one or more composites or related indicators of speech production and/or respiration function. In some embodiments, the output comprises an indicator of a physiological condition such as a respiratory tract status or condition (e.g., congestion or respiratory status with respect to smoking cessation). For example, the output may comprise a clinical rating scale associated with the respiratory tract status, function, or condition. The clinical rating scale may be a commonly used rating scale associated with respiratory tract function, for example, a rating scale associated with severity of congestion. In some embodiments, a trained model is used to evaluate the extracted features corresponding to speech production and/or respiration change/status to generate an output comprising one or more composites or perceptual dimensions. In some embodiments, the output comprises a clinical rating scale. For example, a machine learning algorithm may be used to train or generate a model configured to receive extracted features (and in some cases, one or more composites alone or together with one or more extracted features), optionally with additional data (e.g., sensor data, ambient light level, time of day, etc.) and generate a predicted clinical rating scale. The training data used to generate such models may be audio input that has been evaluated to provide the corresponding clinical rating scale.

The systems, devices, and methods disclosed herein may implement or utilize a plurality or chain or sequence of models or algorithms for performing analysis of the features extracted from a speech or audio signal. In some cases, this process is an example of the process of comparing the multi-dimensional statistical signature to a baseline (e.g., using model/algorithm to evaluate new input data in which the model/algorithm has been trained on past data). In some embodiments, the plurality of models comprises multiple models individually configured to generate specific composites or perceptual dimensions. In some embodiments, one or more outputs of one or more models serve as input for one or more next models in a sequence or chain of models. In some embodiments, one or more features and/or one or more composites are evaluated together to generate an output. In some embodiments, a machine learning algorithm or ML-trained model (or other algorithm) is used to analyze a plurality of feature or feature measurements/metrics extracted from the speech or audio signal to generate an output such as a composite. In some embodiments, the output (e.g., a composite) is used as an input together with other composite(s) and/or features (e.g., metrics that are used to determine composite(s)) that is evaluated by another algorithm configured to generate another output. This output may be a synthesized output incorporating a plurality of composites or one or more composites optionally with additional features that correspond to a readout associated with a physiological condition, status, outcome, or change (e.g., congestion, smoking cessation, etc.). Accordingly, the multi-dimensional signature may include a plurality of features/feature measurements useful for evaluating a perceptual dimension (e.g., articulation, phonation, prosody, etc.), but in some instances, may also include a combination of features with perceptual dimensions. As an illustrative example, a first model (e.g., articulation model) may receive as input extracted audio features and generate a score for articulation. A second model (e.g., prosody model) may receive as input extracted audio features and generate a score for prosody. The first and second models may operate in parallel. A third model may then receive the articulation score and prosody score generated by the first and second models, respectively, and combine or synthesize them into a new output indicative of respiratory tract function or health (e.g., associated with congestion or smoking cessation).

In some embodiments, the systems, devices, and methods disclosed herein combine the features to produce one or more composites that describe or correspond to an outcome, estimation, or prediction. Examples of the outcome, estimation, or prediction can include respiratory tract function such as, for example, a state of congestion or decongestion. Other examples include smoking status (e.g., currently smoking, ceased smoking, length of period smoking or not smoking, etc.). In the case of respiratory tract health, the systems, devices, and methods disclosed herein can include detection of onset of respiratory tract issues/symptoms/problems/health effects or to track the same. In the case of smoking, the systems, devices, and methods disclosed herein can include detection of changes following smoking cessation, the impact of smoking on various parameters (e.g., composites that describe vocal quality, speech quality, and respiratory function), or as endpoints in smoking cessation scenarios.

### Panels of Speech and Acoustic Features

The systems, devices, and methods disclosed herein utilize panel(s) comprising speech and/or acoustic features for evaluating or assessing speech or audio to generate outputs that may correspond to various outcomes. The acoustic features can be used to determine composites such as respiration, phonation, articulation, prosody, or any combination thereof. In some embodiments, the systems, devices, and methods disclosed herein generate an output comprising one composite, two composites, three composites, or four composites, wherein the composite(s) are optionally selected from respiration, phonation, articulation, and prosody. In some embodiments, various acoustic features are used to generate an output using an algorithm or model (e.g., as implemented via the signal processing and evaluation circuitry 110). In some cases, one or more features, one or more composites, or a combination of one or more features and one or more composites are provided as input to an algorithm or model for generating a predicted clinical rating scale corresponding to the physiological status, condition, or change. Various clinical rating scales are applicable. For example, congestion may have a clinical rating scale such as congestion score index (CSI)

Respiration: Respiration can be evaluated using various respiratory measures such as pause rate, loudness, decay of loudness, or any combination thereof. To produce speech, one must first inhale and then generate a positive pressure within the lungs to create an outward flow of air. This air pressure must be of sufficient strength and duration to power the speech apparatus to produce the desired utterance at the desired loudness. Frequent pausing to inhale leads to changes in pause rate; reduced respiratory drive or control can manifest as either reduced loudness or rapid decay of loudness during speech.

Phonation: Phonation can be evaluated using various phonatory measures such as vocal quality, pitch range, or any combination thereof. These measures are modulated by the vocal folds, which are situated in the middle of the larynx. Their vibration—which is set into motion by the column of outward flowing air generated in the lungs—produces the voice. Changes in the rate of vibration (frequency) corresponds with the voice's pitch; changes the air pressure that is allowed to build up beneath the vocal folds corresponds with the voice's loudness (amplitude); changes in vibratory characteristics of the vocal folds correspond with the voice's quality (e.g., breathiness, stridency, etc). Many conditions and diseases have a direct and well-characterized impact on pitch characteristics and vocal quality.

Articulation: Articulation can be evaluated using various articulatory measures such as articulatory precision, speaking rate, or both. These measures provide information about how well the articulators (lips, jaw, tongue, and facial muscles) act to shape and filter the sound. Constrictions made by the lips and tongue create turbulence to produce sounds like “s” and “v,” or to stop the airflow altogether to create bursts as in “t” and “b.” Vowels are produced with a relatively open vocal tract, in which movement of the jaw, tongue and lips create cavity shapes whose resonance patterns are associated with different vowels. In addition, speech sounds can be made via the oral cavity (e.g. the sounds “p, t, b”) or by creating vibratory resonance in the nasal cavity (e.g., “m, n, and ing”). Acoustic analysis, including articulatory precision and speaking rate, can be used to study the features associated with consonants and vowels in healthy- and in disordered-populations because slowness and weakness of articulators impacts both the rate at which speech can be produced, and the ability to create distinct vocal tract shapes (reducing articulatory precision).

Prosody: prosody refers to the rhythm and melody of the outward flow of speech, and can be evaluated or characterized by pitch range, loudness, or both when calculated across a sentence. Conditions such as Parkinson's disease, for example, commonly impact speech prosody. A narrower pitch range makes speech in PD sound monotonous, and reduced loudness makes it sound less animated.

In some embodiments, the one or more composites comprise velopharyngeal function or other perceptual dimensions.

Further detail as to non-limiting embodiments of the composites and the features (and their measures) that correspond to said composites are shown in Table 1.

TABLE 1
physiology and sensorimotor control (e.g., composites), acoustic manifestations
of these composites, the feature measurements that can be used to evaluate these
composites, and sample metrics/measurements are shown.

| | | | Sample |
|---|---|---|---|
| Physiology and | Acoustic | | Interpretable |
| Sensorimotor Control | Manifestations | Measures to Evaluate | Metrics |
| Respiration is the | Reduced respiratory | measures related to | Respiratory |
| driver of speech | drive and control affect | overall loudness in | Drive and |
| production. Moving air | vocal fold vibratory | sustained phonation | Control |
| from the lungs through | characteristics, | and sentences | Metrics: |
| the vocal tract requires | phonatory loudness | measures related to | Phonation |
| the muscular control to | and loudness | loudness decay over | duration, mean |
| inhale (to vital | modulation, and | time in sustained | loudness, |
| capacities above resting | utterance length. | phonation and | loudness decay |
| expiratory level) and to | | Buttercup | Breath group |
| modulate inspiratory | | measures related to | length, # |
| and expiratory | | reduced drive to vocal | pauses, pause |
| muscular contraction to | | fold vibration in | durations |
| control phonatory | | sustained phonation | |
| loudness, | | such as % voicing | |
| | | measures related to | |
| | | duration of sustained | |
| | | phonation or syllables | |
| | | per breath group | |
| Phonation requires | Hypophonia manifests | measures of vocal | Phonatory |
| adequate medialization | as breaks in phonation, | quality such as HNR in | Drive and |
| of the vocal folds to | devoicing, and | sustained phonation | Control |
| allow for the buildup of | breathiness; also can | measures of F0, mean, | Metrics: |
| air pressure beneath | cause reduced | SD, range in sustained | Vocal quality |
| them and blow them | utterance length due to | phonation and sentence | Phonation |
| apart allowing for | air wastage | production | Duration |
| vibration to begin and | Hyperphonia manifests | sustained phonation | Habitual pitch |
| be sustained. [Too little | as strident, strained- | duration (short for | Pitch and |
| medialization and/or | strangled vocal quality, | hypophonia; long for | loudness |
| drive results in | poor pitch and | hyperphonia) | modulation |
| hypophonia; too much | loudness modulation | % voicing in sentence | |
| results in | | production | |
| hyperphonia]. | | | |
| Modulation of pitch | | | |
| and loudness requires | | | |
| synergistic interplay | | | |
| among intrinsic and | | | |
| extrinsic laryngeal | | | |
| musculature to affect | | | |
| changes in vocal fold | | | |
| length, cross-sectional | | | |
| area, longitudinal | | | |
| tension and stiffness. | | | |
| Articulation | Weakness may result in | Buttercup articulation | Articulatory |
| requires coordination | poor articulatory | rate, variation | Precision and |
| among upper | contacts, allowing for | articulatory entropy in | Control |
| articulators (tongue, | air to escape adding | Buttercup and | Metrics: |
| lips, jaw face); velum | noise to the speech | Sentences | speaking rate |
| and pharynx for | signal and reducing the | % voicing in | articulation |
| nasality modulation; | center of spectral | sentences | rate |
| and laryngeal and | energy; spirantization | EMS and LTAS | articulatory |
| respiratory muscles for | during stop closures; | Vowel space area | precision |
| production of voiced | reduced center | | vowel space |
| and voiceless sounds. | frequency and | | area |
| The pattern of | increased duration of | | |
| neurological | fricative and affricates; | | |
| breakdown can be | VOT abnormalities; | | |
| inferred from the | nasalization spread to | | |
| aspects of articulation | nonnasal sounds. | | |
| that are negatively | Speaking and | | |
| affected. | articulation rate | | |
| | reductions are most | | |
| | common in dysarthria. | | |
| Velopharyngeal | Hypernasality is the | measures of energy | Resonatory |
| function | most common | below 500 Hz in | Precision and |
| refers to the balance of | consequence of | sustained phonation | Control |
| energy amplitude | velopharyngeal | and in Buttercup | Metrics |
| occurring in the oral, | musculature weakness | (mean, slope over | hypernasality |
| oropharyngeal, and | (flaccid and/or spastic | repetitions) | metric |
| nasal cavities during | paralysis). | SNR in Buttercup | nasal air |
| production of voiced | Hypernasality can | overall and over | emission |
| sounds. Oral resonance | affect production of | repetitions to capture | |
| required activation of | consonants (voiced and | nasal emission | |
| velar and pharyngeal | voiceless) that require | | |
| muscles to close off the | impounding oral air | | |
| velopharyngeal port, | pressure (stop | | |
| directing phonatory | consonants in | | |
| energy through the | particular). Nasal air | | |
| mouth only. | emission may be | | |
| Modulation of nasality | audible during speech. | | |
| requires alternation of | Hyponasality in | | |
| velopharyngeal | dysarthrias is less | | |
| musculature activation | common, and less | | |
| and relaxation. | detrimental to | | |
| | articulatory integrity. | | |
| Prosody, the rate and | Prosodic abnormalities | Rhythm metrics, | Prosody |
| rhythm of speech, | include: | EMS, LTAS, VSA | Metrics |
| refers to the modulation | too much or too little | on sentences | Speech |
| of syllable duration, | pitch | | melody |
| pitch, loudness, and | modulation/variation | | Speech |
| distinctiveness during | too much or too little | | loudness |
| connected speech. This | loudness | | variation |
| modulation is produced | modulation/variation | | Speech |
| by the orchestrated | too much or too little | | rhythm |
| interplay among | syllable duration | | |
| respiratory, phonatory | contrastivity | | |
| and articulatory | too much or too little | | |
| musculature. | vowel quality | | |
| | contrastivity | | |

### System and Device Interfaces

In some embodiments, the systems, devices, and methods disclosed herein comprise a user interface for prompting or obtaining an input speech or audio signal, and delivering the output or notification to the user. The user interface may be communicatively coupled to or otherwise in communication with the audio input circuitry 108 and/or notification element 114 of the speech assessment device 102. The speech assessment device can be any suitable electronic device capable of receiving audio input, processing/analyzing the audio, and providing the output signal or notification. Non-limiting examples of the speech assessment device include smartphones, tablets, laptops, desktop computers, and other suitable computing devices.

In some embodiments, the interface comprises a touchscreen for receiving user input and/or displaying an output or notification associated with the output. In some cases, the output or notification is provided through a non-visual output element such as, for example, audio via a speaker. The audio processing and analytics portions of the instant disclosure are provided via computer software or executable instructions. In some embodiments, the computer software or executable instructions comprise a computer program, a mobile application, or a web application or portal. The computer software can provide a graphic user interface via the device display. The graphic user interface can include a user login portal with various options such as to input or upload speech/audio data/signal/file, review current and/or historical speech/audio inputs and outputs (e.g., analyses), and/or send/receive communications including the speech/audio inputs or outputs.

In some embodiments, the user is able to configure the software based on a desired physiological status the user wants to evaluate or monitor. For example, the user may select smoking/smoking cessation to configure the software to utilize the appropriate algorithms for determining speech production and/or respiration status associated with smoking status/cessation. The software may then actively and/or passively collect speech data for the user and monitor speech production and/or respiration status as an indicator of smoking status over time. In some embodiments, the graphic user interface provides graphs, charts, and other visual indicators for displaying the status or progress of the user with respect to the physiological status or condition, for example, smoking cessation.

Alternatively, or in combination, the physiological status can be congestion/decongestion. In some embodiments, a user who is experiencing speech/respiration-related health issues or physiological conditions (e.g., respiratory tract condition such as congestion) is able to utilize the user interface to configure the computer program for evaluating and/or monitoring the particular respiratory tract condition such as congestion using speech production and/or respiration status metrics (e.g., using composites attributable to congestion status).

In some embodiments, the computer software is a mobile application and the device is a smartphone. This enables a convenient, portable mechanism to monitor physiological status (e.g., respiratory tract condition or health) as related to speech and/or respiration without requiring the user to be in the clinical setting. In some embodiments, he mobile application includes a graphic user interface allowing the user to login to an account, review current and historical speech analysis results, and visualize the results over time. For example, graphs and timelines showing improvement in respiration and speech production metrics over time following smoking cessation or an overall positive trend associated with improved congestion over time while a user is recovering from an illness that triggered the congestion.

In some embodiments, the device and/or software is configured to securely transmit the results of the speech analysis to a third party (e.g., healthcare provider of the user). In some embodiments, the user interface is configured to provide performance metrics associated with the physiological or health condition (e.g., respiratory tract health). In this case, statistical measures of long-term success are displayed for the user based on the length of time the user has ceased smoking.

In some embodiments, when a status change or significant increase/decrease in speech production and/or respiration status is detected, the device or interface displays a warning or other message to the user based on the change. For example, a deterioration in speech production and/or respiration quality may be detected when a user relapses from smoking cessation. A sudden shift in the speech/respiration metrics can trigger detection of a status change. In this example, the device may detect a deterioration or decrease in speech production quality and respiration status, and subsequently displays a warning message to the user requesting confirmation of the status change and providing advice on how to deal with the status change.

### Machine Learning Algorithms

In some embodiments, the systems, devices, and methods disclosed herein utilize one or algorithms or models configured to evaluate or assess speech and/or respiration, which may include generating an output indicative of a physiological state or condition or change (e.g., congestion, smoking cessation, etc.) corresponding to the speech and/or respiration evaluation. In some embodiments, the systems, devices, and methods disclosed herein utilize one or more machine learning algorithms or models trained using machine learning to evaluate or assess speech and/or respiration. In some cases, one or more algorithms are used to process raw speech or audio data (e.g., diarization). The algorithm(s) used for speech processing may include machine learning and non-machine learning algorithms. In some cases, one or more algorithms are used to extract or generate one or more measures of features useful for generating or evaluating a perceptual dimension or composite (e.g., articulation, prosody, etc). The extracted feature(s) may be input into an algorithm or ML-trained model to generate an output comprising one or more composites or perceptual dimensions. In some embodiments, one or more features, one or more composites, or a combination of one or more features and one or more composites are provided as input to a machine learning algorithm or ML-trained model to generate the desired output. In some embodiments, the output comprises another composite or perceptual dimension. In some embodiments, the output comprises an indicator of a physiological condition such as a respiratory tract status or condition (e.g., congestion). For example, the output may comprise a clinical rating scale associated with the respiratory tract status, function, or condition. The clinical rating scale may be a commonly used rating scale associated with respiratory tract function, for example, a rating scale associated with severity of congestion.

In some embodiments, the signal processing and evaluation circuitry comprises one or more machine learning modules comprising machine learning algorithms or ML-trained models for evaluating the speech or audio signal, the processed signal, the extracted features, or the extracted composite(s) or a combination of features and composite(s). A machine learning module may be trained on one or more training data sets. A machine learning module may include a model trained on at least about: 1, 2, 3, 4, 5, 6, 7, 8, 9, 10, 20, 30, 40, 50, 60, 70, 80, 90, 100 data sets or more (e.g., speech/audio signals). A machine learning module may be validated with one or more validation data sets. A validation data set may be independent from a training data set. The machine learning module(s) and/or algorithms/models disclosed herein can be implemented using computing devices or digital process devices or processors as disclosed herein.

A machine learning algorithm may use a supervised learning approach. In supervised learning, the algorithm can generate a function or model from training data. The training data can be labeled. The training data may include metadata associated therewith. Each training example of the training data may be a pair consisting of at least an input object and a desired output value (e.g., a composite score). A supervised learning algorithm may require the individual to determine one or more control parameters. These parameters can be adjusted by optimizing performance on a subset, for example a validation set, of the training data. After parameter adjustment and learning, the performance of the resulting function/model can be measured on a test set that may be separate from the training set. Regression methods can be used in supervised learning approaches.

A machine learning algorithm may use an unsupervised learning approach. In unsupervised learning, the algorithm may generate a function/model to describe hidden structures from unlabeled data (e.g., a classification or categorization that cannot be directly observed or computed). Since the examples given to the learner are unlabeled, there is no evaluation of the accuracy of the structure that is output by the relevant algorithm. Approaches to unsupervised learning include clustering, anomaly detection, and neural networks.

A machine learning algorithm is applied to patient data to generate a prediction model. In some embodiments, a machine learning algorithm or model may be trained periodically. In some embodiments, a machine learning algorithm or model may be trained non-periodically.

As used herein, a machine learning algorithm may include learning a function or a model. The mathematical expression of the function or model may or may not be directly computable or observable. The function or model may include one or more parameter(s) used within a model. In some embodiments, a machine learning algorithm comprises a supervised or unsupervised learning method such as, for example, support vector machine (SVM), random forests, gradient boosting, logistic regression, decision trees, clustering algorithms, hierarchical clustering, K-means clustering, or principal component analysis. Machine learning algorithms may include linear regression models, logistical regression models, linear discriminate analysis, classification or regression trees, naive Bayes, K-nearest neighbor, learning vector quantization (LVQ), support vector machines (SVM), bagging and random forest, boosting and Adaboost machines, or any combination thereof. In some embodiments, machine learning algorithms include artificial neural networks with non-limiting examples of neural network algorithms including perceptron, multilayer perceptrons, back-propagation, stochastic gradient descent, Hopfield network, and radial basis function network. In some embodiments, the machine learning algorithm is a deep learning neural network. Examples of deep learning algorithms include convolutional neural networks (CNN), recurrent neural networks, and long short-term memory networks.

### Digital Processing Device

The systems, devices, and methods disclosed herein may be implemented using a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

A digital processing device as described herein either includes or is operatively coupled to a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

A system or method as described herein can be used to generate, determine, and/or deliver a degree of haptic feedback which may then be used to determine whether a subject value falls within or outside of a threshold value. In addition, in some embodiments, a system or method as described herein generates a database as containing or comprising one or more haptic feedback degrees. In some embodiments, a database herein provides a relative risk of presence/absence of a status (outcome) associated with haptic feedback that fall either within or outside of a threshold value.

Some embodiments of the systems described herein are computer based systems. These embodiments include a CPU including a processor and memory which may be in the form of a non-transitory computer-readable storage medium. These system embodiments further include software that is typically stored in memory (such as in the form of a non-transitory computer-readable storage medium) where the software is configured to cause the processor to carry out a function. Software embodiments incorporated into the systems described herein contain one or more modules.

In various embodiments, an apparatus comprises a computing device or component such as a digital processing device. In some of the embodiments described herein, a digital processing device includes a display to send visual information to a user. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information from a user. Non-limiting examples of input devices suitable for use with the systems and methods described herein include a keyboard, a mouse, trackball, track pad, or stylus. In some embodiments, the input device is a touch screen or a multi-touch screen.

### Non-Transitory Computer-Readable Storage Medium

The systems and methods described herein typically include one or more non-transitory (non-transient) computer-readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer-readable storage medium is optionally removable from a digital processing device. In some embodiments, a computer-readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Programs

Typically the systems and methods described herein include at least one computer program, or use of the same. A computer program includes a sequence of instructions, executable in the digital processing device's CPU, written to perform a specified task. Computer-readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer-readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Mobile Application

In some embodiments, a computer program includes a mobile application provided to a mobile electronic device. In some embodiments, the mobile application is provided to a mobile electronic device at the time it is manufactured. In other embodiments, the mobile application is provided to a mobile electronic device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g. not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable compiled applications.

### Software Module

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

Although the detailed description contains many specifics, these should not be construed as limiting the scope of the disclosure but merely as illustrating different examples and aspects of the present disclosure. It should be appreciated that the scope of the disclosure includes other embodiments not discussed in detail above. Various other modifications, changes and variations which will be apparent to those skilled in the art may be made in the arrangement, operation and details of the method and apparatus of the present disclosure provided herein without departing from the spirit and scope of the invention as described herein. For example, one or more aspects, components or methods of each of the examples as disclosed herein can be combined with others as described herein, and such modifications will be readily apparent to a person of ordinary skill in the art. For each of the methods disclosed herein, a person of ordinary skill in the art will recognize many variations based on the teachings described herein. The steps may be completed in a different order. Steps may be added or deleted. Some of the steps may comprise sub-steps of other steps. Many of the steps may be repeated as often as desired, and the steps of the methods can be combined with each other.

### EXAMPLES

### Example 1

### Smoking Cessation

A user smartphone is programmed with a mobile application that utilizes the smartphone's microphone to passively record speech by the user. During setup, the mobile application prompts the user to vocalize speech that is then detected by the microphone and recorded to the phone's memory. This speech is used to generate an initial or baseline statistical signature of speech production and respiration status. The baseline statistical signature is generated by processing the speech audio signal, including a quality control check to ensure adequate signal-to-noise ratio. The speech signal is saved locally and backed up to a server on the cloud. The speech audio is diarized and transcribed using diarization and transcription algorithms. The transcribed text is aligned to the speaker timepoints, resulting in a text-acoustic alignment dataset. Specific speech and respiration features are extracted from the text-acoustic data to generate measures for features useful for predicting or estimating perceptual dimensions/composites including respiration, phonation, articulation, and prosody. These measured features are then entered as input to a machine learning-trained neural network configured to generate an output corresponding to smoking status or smoking cessation status. Additional features are optionally taken into consideration including sensor data (e.g., vital sign data such as heart rate/blood pressure from a fitness tracker), time of day, ambient light levels, smartphone usage pattern, and/or user input.

In this case, the user has decided to quit smoking and downloaded the mobile application to keep track of physiological changes associated with smoking/cessation of smoking that can be monitored via corresponding changes in speech production and respiration status/ability. The baseline is taken on the first day the user decides to quit smoking. The mobile application actively prompts the user to provide speech samples on a daily basis to enable continued monitoring of speech/respiration over time. The mobile application also has a passive mode setting that the user has turned on to enable passive collection of speech to supplement the active prompts for speech tasks which the user sometimes neglects.

The smartphone is used to analyze speech samples and track the analysis results over time. The mobile application includes a graphic user interface allowing the user to login to an account, review current and historical speech analysis results, and visualize the results over time (e.g., graphs and timelines showing improvement in respiration and speech production metrics over time following smoking cessation). The mobile application also provides the option to securely transmit the results to a third party (e.g., healthcare provider). In this case, the user shares the information with his doctor who is helping to monitor his health during his attempt to quit smoking. The mobile application interface also provides related performance metrics associated with the attempt to quit smoking. In this case, statistical measures of long-term success are displayed for the user based on the length of time the user has ceased smoking.

When the user relapses and resumes smoking for several days during this time period, the mobile application's algorithms detect a deterioration or decrease in speech production quality and respiration status. The mobile application then displays a warning message to the user requesting confirmation of relapse and providing advice on how to cope with relapse. With the aid of the smartphone mobile application, the user is able to resume smoking cessation and eventually quit for good.

### Example 2

### Congestion

A user utilizes the smartphone programmed with the mobile application of Example 1. In this case, the user has recently become sick and is experiencing congestion symptoms. Accordingly, the user configures the mobile application to collect speech that is used to generate an initial or baseline statistical signature of speech production and respiration status as it pertains to congestion status. The speech audio is diarized and transcribed using diarization and transcription algorithms. The transcribed text is aligned to the speaker timepoints, resulting in a text-acoustic alignment dataset. Specific speech and respiration features are extracted from the text-acoustic data to generate measures for features useful for predicting or estimating perceptual dimensions/composites including respiration, phonation, articulation, and prosody. These measured features are then entered as input to a machine learning-trained neural network configured to generate an output corresponding to congestion status (e.g., a clinical rating scale for congestion). Additional features are optionally taken into consideration including sensor data (e.g., vital sign data such as heart rate/blood pressure from a fitness tracker), time of day, ambient light levels, smartphone usage pattern, and/or user input.

The mobile application actively prompts the user to provide speech samples on a daily basis to enable continued monitoring of speech/respiration over time. The mobile application also has a passive mode setting that the user has turned on to enable passive collection of speech to supplement the active prompts for speech tasks which the user sometimes neglects.

The smartphone is used to analyze speech samples and track the analysis results over time. The mobile application includes a graphic user interface allowing the user to login to an account, review current and historical speech analysis results, and visualize the results over time (e.g., graphs and timelines showing improvement in respiration and speech production metrics over time as well as the predicted congestion status). The mobile application also provides the option to securely transmit the results to a third party (e.g., healthcare provider). In this case, the user shares the information with his doctor who is helping to monitor his illness. After a few days, the mobile application shows that the user's congestion status is improving. This information is transmitted to the user's doctor who advises cancelling a follow-up appointment as the symptoms (including congestion) are rapidly disappearing.

### Example 3

### Respiratory Tract Function

A user utilizes the smartphone programmed with the mobile application of Example 1. In this case, the user has long experienced respiration problems including allergies, emphysema, and other respiratory tract health problems throughout his life. Accordingly, the user uses this mobile application to monitor respiratory tract function as he makes lifestyle changes to address his health problems. Accordingly, the user configures the mobile application to collect speech that is used to generate an initial or baseline statistical signature of speech production and respiration status as it pertains to respiratory tract health or function. The speech audio is diarized and transcribed using diarization and transcription algorithms. The transcribed text is aligned to the speaker timepoints, resulting in a text-acoustic alignment dataset. Specific speech and respiration features are extracted from the text-acoustic data to generate measures for features useful for predicting or estimating perceptual dimensions/composites including respiration, phonation, articulation, and prosody. These measured features are then entered as input to a machine learning-trained neural network configured to generate an output corresponding to respiratory tract status (e.g., a rating scale associated with respiratory tract function). Additional features are optionally taken into consideration including sensor data (e.g., vital sign data such as heart rate/blood pressure from a fitness tracker), time of day, ambient light levels, smartphone usage pattern, and/or user input.

The mobile application actively prompts the user to provide speech samples on a daily basis to enable continued monitoring of speech/respiration over time. The mobile application also has a passive mode setting that the user has turned on to enable passive collection of speech to supplement the active prompts for speech tasks which the user sometimes neglects.

The smartphone is used to analyze speech samples and track the analysis results over time. The mobile application includes a graphic user interface allowing the user to login to an account, review current and historical speech analysis results, and visualize the results over time (e.g., graphs and timelines showing improvement in respiration and speech production metrics over time as well as the predicted respiratory tract function). The mobile application also provides the option to securely transmit the results to a third party (e.g., healthcare provider). The user continues to adjust his lifestyle, including moving to a different location that is cool and dry with low humidity. These lifestyle changes turn out to be effective, which is reflected in the respiratory tract function/health status metrics steadily improving over a period of several months.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for assessing speech changes resulting from respiratory tract function, the device comprising:
audio input circuitry configured to provide an input signal that is indicative of speech provided by a user;
signal processing circuitry configured to:
receive the input signal;
process the input signal to generate an instantaneous multi-dimensional statistical signature of speech production abilities of the user, and compare the multi-dimensional statistical signature against one or more baseline statistical signatures of speech production ability derived or obtained from the user; and
provide a speech change identification signal attributable to respiratory tract function of the user, based on the multi-dimensional statistical signature comparison; and
a notification element coupled to the signal processing circuitry, the notification element configured to receive the speech change identification and provide at least one notification signal to the user.

2. The device of claim 1, wherein the multi-dimensional statistical signature spans one or more of the following perceptual dimensions: articulation, prosodic variability, phonation changes, rate, and rate variation.

3. The device of claim 1, wherein the signal processing circuitry is configured to process the input signal by measuring speech features represented in the input signal, the speech features comprising one or more of articulation rate, articulation entropy, vowel space area, energy decay slope, phonatory duration, and average pitch.

4. The device of claim 3, wherein the signal processing circuitry is configured to compare the multi-dimensional statistical signature against the one or more baseline statistical signatures of speech production ability by comparing each speech feature to a corresponding baseline speech feature of the one or more baseline statistical signatures of speech production ability.

5. The device of claim 1, wherein the signal processing circuitry is configured to process the input signal utilizing the input signal and additional data comprising one or more of sensor data, a time of day, an ambient light level, a device usage pattern of the user, or a user input.

6. The device of claim 5, wherein the signal processing circuitry is configured to process the input signal by selecting or adjusting the one or more baseline statistical signatures of speech production ability based on the additional data.

7. The device of claim 1, wherein the device is a mobile computing device operating an application for assessing speech changes resulting from respiratory tract function.

8. The device of claim 7, wherein the application queries the user periodically to provide a speech sample from which the input signal is derived.

9. The device of claim 7, wherein the application facilitates the user spontaneously providing a speech sample from which the input signal is derived.

10. The device of claim 7, wherein the application passively detects changes in speech patterns of the user and initiates generation of the instantaneous multi-dimensional statistical signature of speech production abilities of the user.

11. The device of claim 1, wherein the notification element comprises a display.

12. The device of claim 11, wherein the signal processing circuitry is further configured to cause the display to prompt the user to provide a speech sample from which the input signal is derived.

13. The device of claim 12, wherein the at least one notification signal comprises a display notification instructing the user to take action to relieve symptoms associated with respiratory tract function.

14. A method for assessing speech changes resulting from respiratory tract function, the method comprising:
receiving an input signal that is indicative of speech provided by a user;
extracting a multi-dimensional statistical signature of speech production abilities of the user from the input signal;
comparing the multi-dimensional statistical signature against one or more baseline statistical signatures of speech production ability; and
providing a speech change identification signal attributable to respiratory tract function of the user, based on the multi-dimensional statistical signature comparison.

15. The method of claim 14, wherein the one or more baseline statistical signatures of speech production ability are derived or obtained from the user.

16. The method of claim 14, wherein the one or more baseline statistical signatures of speech production ability are at least partially based on normative acoustic data from a database.

17. The method of claim 14, wherein the comparing the multi-dimensional statistical signature against the one or more baseline statistical signatures of speech production ability comprises applying a machine learning algorithm to the multi-dimensional statistical signature.

18. The method of claim 17, wherein the machine learning algorithm is trained with past comparisons for other users.

19. The method of claim 14, wherein:
extracting the multi-dimensional statistical signature of speech production abilities of the user from the input signal comprises measuring speech features across one or more of the following perceptual dimensions: articulation, prosodic variability, phonation changes, rate, and rate variation; and
comparing the multi-dimensional statistical signature against the one or more baseline statistical signatures of speech production ability comprises comparing each speech feature to a corresponding baseline speech feature of the one or more baseline statistical signatures of speech production ability.

20. A non-transitory computer readable storage medium which, when executed by a computer, causes the computer to:
receive an input signal that is indicative of speech provided by a user;
extract a multi-dimensional statistical signature of speech production abilities of the user from the input signal;
compare the multi-dimensional statistical signature against one or more baseline statistical signatures of speech production ability; and
provide a speech change identification signal attributable to respiratory tract function of the user, based on the multi-dimensional statistical signature comparison.

21. A device for assessing speech production and respiration changes after smoking cessation, the device comprising:
audio input circuitry configured to provide an input signal that is indicative of speech and respiration provided by a user;
signal processing circuitry configured to:
receive the input signal;
process the input signal to generate an instantaneous multi-dimensional statistical signature of speech production and respiration abilities of the user, and compare the multi-dimensional statistical signature against one or more baseline statistical signatures of speech production and respiration ability derived or obtained from the user; and
provide a speech production and respiration change identification signal based on the multi-dimensional statistical signature comparison; and
a notification element coupled to the signal processing circuitry, the notification element configured to receive the speech production and respiration identification signal and provide at least one notification signal to the user.

22. The device of claim 21, wherein the multi-dimensional statistical signature spans one or more of the following perceptual dimensions: articulation, prosodic variability, phonation changes, rate, and rate variation.

23. The device of claim 21, wherein the signal processing circuitry is configured to process the input signal by measuring speech features represented in the input signal, the speech features comprising one or more of speaking rate, articulation rate, articulation entropy, vowel space area, energy decay slope, phonatory duration, and average pitch.

24. The device of claim 23, wherein the signal processing circuitry is configured to compare the multi-dimensional statistical signature against the one or more baseline statistical signatures of speech production ability by comparing each speech feature to a corresponding baseline speech feature of the one or more baseline statistical signatures of speech production ability.

25. The device of claim 21, wherein the signal processing circuitry is configured to process the input signal utilizing the input signal and additional data comprising one or more of sensor data, a time of day, an ambient light level, a device usage pattern of the user, or a user input.

26. The device of claim 25, wherein the signal processing circuitry is configured to process the input signal by selecting or adjusting the one or more baseline statistical signatures of speech production ability based on the additional data.

27. The device of claim 21, wherein the device is a mobile computing device operating an application for assessing speech production and respiration changes after smoking cessation.

28. The device of claim 27, wherein the application queries the user periodically to provide a speech sample from which the input signal is derived.

29. The device of claim 27, wherein the application facilitates the user spontaneously providing a speech sample from which the input signal is derived.

30. The device of claim 27, wherein the application passively detects changes in speech patterns of the user and initiates generation of the instantaneous multi-dimensional statistical signature of speech production abilities of the user.

31. The device of claim 21, wherein the notification element comprises a display.

32. The device of claim 31, wherein the signal processing circuitry is further configured to cause the display to prompt the user to provide a speech sample from which the input signal is derived.

33. A method for assessing speech production and respiration changes after smoking cessation, the method comprising:
receiving an input signal that is indicative of speech production and respiration provided by a user;
extracting a multi-dimensional statistical signature of speech production and respiration abilities of the user from the input signal;
comparing the multi-dimensional statistical signature against one or more baseline statistical signatures of speech production and respiration ability; and
providing a speech production and respiration change identification signal attributable to smoking cessation based on the multi-dimensional statistical signature comparison.

34. The method of claim 33, wherein the one or more baseline statistical signatures of speech production and respiration abilities are derived or obtained from the user.

35. The method of claim 33, wherein the one or more baseline statistical signatures of speech production and respiration abilities are at least partially based on normative acoustic data from a database.

36. The method of claim 33, wherein the comparing the multi-dimensional statistical signature against the one or more baseline statistical signatures of speech production and respiration abilities comprises applying a machine learning algorithm to the multi-dimensional statistical signature.

37. The method of claim 36, wherein the machine learning algorithm is trained with past comparisons for other users.

38. The method of claim 33, wherein:
extracting the multi-dimensional statistical signature of speech production abilities of the user from the input signal comprises measuring speech features across one or more of the following perceptual dimensions: articulation precision, respiratory support, nasality, prosody, and phonatory control; and
comparing the multi-dimensional statistical signature against the one or more baseline statistical signatures of speech production ability comprises comparing each speech feature to a corresponding baseline speech feature of the one or more baseline statistical signatures of speech production ability.

39. A non-transitory computer readable storage medium which, when executed by a computer, causes the computer to:
receive an input signal that is indicative of speech production and respiration provided by a user;
extract a multi-dimensional statistical signature of speech production and respiration abilities of the user from the input signal;
compare the multi-dimensional statistical signature against one or more baseline statistical signatures of speech production and respiration abilities; and
provide a speech production and respiration change identification signal attributable to smoking cessation of the user, based on the multi-dimensional statistical signature comparison.

40. The non-transitory computer readable storage medium of claim 39, wherein the computer is a smartphone.

41. A device for assessing speech changes resulting from congestion state, the device comprising:
audio input circuitry configured to provide an input signal that is indicative of speech provided by a user;
signal processing circuitry configured to:
receive the input signal;
process the input signal to generate an instantaneous multi-dimensional statistical signature of speech production abilities of the user, and compare the multi-dimensional statistical signature against one or more baseline statistical signatures of speech production ability derived or obtained from the user; and
provide a speech change identification signal attributable to congestion state of the user, based on the multi-dimensional statistical signature comparison; and
a notification element coupled to the signal processing circuitry, the notification element configured to receive the speech change identification and provide at least one notification signal to the user.

42. The device of claim 41, wherein the multi-dimensional statistical signature spans one or more of the following perceptual dimensions: articulation, prosodic variability, phonation changes, rate, and rate variation.

43. The device of claim 41, wherein the signal processing circuitry is configured to process the input signal by measuring speech features represented in the input signal, the speech features comprising one or more of articulation rate, articulation entropy, vowel space area, energy decay slope, phonatory duration, and average pitch.

44. The device of claim 43, wherein the signal processing circuitry is configured to compare the multi-dimensional statistical signature against the one or more baseline statistical signatures of speech production ability by comparing each speech feature to a corresponding baseline speech feature of the one or more baseline statistical signatures of speech production ability.

45. The device of claim 41, wherein the signal processing circuitry is configured to process the input signal utilizing the input signal and additional data comprising one or more of sensor data, a time of day, an ambient light level, a device usage pattern of the user, or a user input.

46. The device of claim 45, wherein the signal processing circuitry is configured to process the input signal by selecting or adjusting the one or more baseline statistical signatures of speech production ability based on the additional data.

47. The device of claim 41, wherein the device is a mobile computing device operating an application for assessing speech changes resulting from congestion state.

48. The device of claim 47, wherein the application queries the user periodically to provide a speech sample from which the input signal is derived.

49. The device of claim 47, wherein the application facilitates the user spontaneously providing a speech sample from which the input signal is derived.

50. The device of claim 47, wherein the application passively detects changes in speech patterns of the user and initiates generation of the instantaneous multi-dimensional statistical signature of speech production abilities of the user.

51. The device of claim 41, wherein the notification element comprises a display.

52. The device of claim 51, wherein the signal processing circuitry is further configured to cause the display to prompt the user to provide a speech sample from which the input signal is derived.

53. The device of claim 52, wherein the at least one notification signal comprises a display notification instructing the user to take action to relieve congestion symptoms.

54. A method for assessing speech changes resulting from congestion state, the method comprising:
receiving an input signal that is indicative of speech provided by a user;
extracting a multi-dimensional statistical signature of speech production abilities of the user from the input signal;
comparing the multi-dimensional statistical signature against one or more baseline statistical signatures of speech production ability; and
providing a speech change identification signal attributable to congestion state of the user, based on the multi-dimensional statistical signature comparison.

55. The method of claim 54, wherein the one or more baseline statistical signatures of speech production ability are derived or obtained from the user.

56. The method of claim 54, wherein the one or more baseline statistical signatures of speech production ability are at least partially based on normative acoustic data from a database.

57. The method of claim 54, wherein the comparing the multi-dimensional statistical signature against the one or more baseline statistical signatures of speech production ability comprises applying a machine learning algorithm to the multi-dimensional statistical signature.

58. The method of claim 57, wherein the machine learning algorithm is trained with past comparisons for other users.

59. The method of claim 54, wherein:
extracting the multi-dimensional statistical signature of speech production abilities of the user from the input signal comprises measuring speech features across one or more of the following perceptual dimensions: articulation, prosodic variability, phonation changes, rate, and rate variation; and
comparing the multi-dimensional statistical signature against the one or more baseline statistical signatures of speech production ability comprises comparing each speech feature to a corresponding baseline speech feature of the one or more baseline statistical signatures of speech production ability.

60. A non-transitory computer readable storage medium which, when executed by a computer, causes the computer to:
receive an input signal that is indicative of speech provided by a user;
extract a multi-dimensional statistical signature of speech production abilities of the user from the input signal;
compare the multi-dimensional statistical signature against one or more baseline statistical signatures of speech production ability; and provide a speech change identification signal attributable to congestion state of the user, based on the multi-dimensional statistical signature comparison.
